(19) United States
(12) Patent Application Publication
(10) Pub. No.: US 2024/0100075 A1
(43) Pub. Date: Mar. 28, 2024

(54) Synthetic Compositions Comprising LNFP III and LSTa

(71) Applicant: FrieslandCampina Nederland B.V., Amersfoort (NL)

(72) Inventor: Vassilios Triantis, Wageningen (NL)

(21) Appl. No.: 18/265,028

(22) PCT Filed: Dec. 15, 2021
(86) PCT No.: PCT/EP2021/085819

§ 371 Date: Jun. 2, 2023

(30) Foreign Application Priority Data
Dec. 16, 2020 (EP) 20214739.3

(51) Int. Cl. A61K 31/702; A61K 35/20; A61K 36/18; A61P 37/04

(52) U.S. Cl. CPC A61K 31/702; A61K 35/20; A61K 36/18; A61P 37/04; A61K 2035/115

(57) ABSTRACT

A synthetic composition comprising a carbohydrate component, a fat component and a protein component, wherein the carbohydrate component comprises lacto-N-fucopentaose III (LNFP III) and sialyllacto-N-tetraose a (LSTa), preferably wherein the weight ratio between LNFP III and LSTa is between 1:100 and 100:1.

### FIELD OF THE INVENTION

The invention disclosed herein relates to a synthetic composition comprising a carbohydrate component, a fat component and a protein component, wherein the carbohydrate component comprises lacto-N-fucopentaose III (LNFP III) and sialyllacto-N-tetraose a (LSTa).

The invention further relates to the use of this synthetic composition for use as a medicine or for us in treating inflammatory disease, or for use in one or more of: upregulating FoxP3, upregulating IL10, upregulation regulatory T cells (Tregs), and downregulation immune responses.

### BACKGROUND

Breast feeding is the best way to ensure healthy growth and development of infants during the first months of life. It is recommended by the WHO to exclusively provide breast feeding during the first six months of life and the introduction of safe and appropriate complementary feeding thereafter to supplement continued breast feeding up to two years of age or beyond. However, when mothers cannot or choose not to breastfeed for whatever reason and a safe alternative to breast feeding is required, there is a legitimate role for breast milk substitutes, produced according to strict international compositional and safety standards.

Processes underlying Th17 cell differentiation and activation, as well as Th17-specific cytokines, chemokines, and transcription factors, have been characterized R. Seki and K. Nishizawa; Biomed Res Clin Prac, 2016 Volume 1(4) pp 126-147). Th17 cells have a central role in maintaining the integrity of mucosal barriers through stimulation of epithelial cell proliferation and induction of tight junction proteins such as claudins (Brewer M G, Yoshida T, Kuo F I et al. Int J Mol Sci 2019; 20(17)), but also contributing to pathogen clearance at mucosal surfaces, by inducing expression of antimicrobial molecules (defensins) (Blaschitz C, Raffatellu M, J Clin Immunol. 2010; 30(2):196-203) and active recruitment of neutrophils though induction of cytokine expression such as G-CSF (CSF3). Their development is dependent on the transcription factor RORγt which is also a marker for differentiating Th17 cells from other T helper subsets. RORγt is driving expression of the Th17 signature cytokine IL17 in humans (Castro G, Liu X, Ngo K, De Leon-Tabaldo A, Zhao S, Luna-Roman R, et al. PLoS One. 2017 Aug. 1; 12(8)).

Since IL-17 is a major player in tissue-specific immune pathology, Th17 cells, a major source of the cytokine, have been a subject of intensive research and have been at the forefront of clinical studies. For example absence of Th17 cells at mucosal surfaces has been linked to microbial translocation and subsequent chronic inflammation. Th17 cells derive from naïve CD4+ T-cells when the latter are exposed to cytokines such as IL6 or IL23 (a heterodimer composed by IL12B and IL23A). The same precursor CD4+ naïve T-cells under different cytokine stimuli give also rise to regulatory T (Treg) cells that stand central in controlling immune responses and Th17 biology. Treg cells are marked by the expression of the transcription factor FoxP3 and high expression of the surface marker CD25 (IL2RA). By producing anti-inflammatory cytokines such as IL10, they dampen immune responses and have a protective role against auto-immune diseases. Given that Th17 cells share common progenitors with Treg cells that, in turn, control Th17 cells, the Treg/Th17 axis is important for fine-tuning the intensity of inflammatory responses. A combination of factors act in synergy to regulate the Th17/Treg balance and inter-Th17 subset balance, and it has been shown that Th17 cells can differentiate to Treg cells during resolution of inflammation (Gagliani N, Vesely M C, Iseppon A et al. 2015 Nature. 523. 221-5). Therapeutic interventions that can tune such balances help increase therapeutic options for many cases of autoimmune and inflammatory diseases and predisposed individuals.

Fine-tuning the balance between regulatory cells and Th17 cells is important, hence there is a need for compositions that have an effect on this balance. The same is true for pathogenic and non-pathogenic subsets of Th17 cells. Local imbalance, likely in the intestine, between such populations causing over-proliferation of Th17 cells results in exacerbation of autoimmunity in remote organs.

From a clinical perspective, the availability of drugs that explicitly act on specific cells or accumulate in local tissues/organs is important, given that systemic drug administration is generally apt to lead to adverse events.

Fine-tuning of Treg/Th17, and of the subsets of Th17 cells, appears important in the intestine, in particular. Clinical trials using IL-17A inhibitors for psoriasis, ankylosing spondylitis, and RA showed promising results. Inhibition of IL-17 activity should lead to susceptibility to infection. IL-17 and IL-22 produced by Th17 are considered to be important for epithelial cell production of 6-defensin that has antifungal activity, and inhibition of this loop may lead to increases in fungi, leading to an enhanced innate immunity response in intestinal mucosa. (Factors regulating Th17 cells: a review; Reiko Seki and Kazuhisa Nishizawa; Biomed Res Clin Prac, 2016 Volume 1(4) pp 126-147; doi: 10.15761/BRCP.1000122).

Johnson et al described that decrease in inflammatory and autoimmune disease susceptibility, which results from commensal microbial immunologic responses, has largely been attributed to CD25+FoxP3+ regulatory T cells (Tregs). Although gut-associated FoxP3− Tregs are well known, the loss of functional FoxP3, whether through murine genetic manipulation or in the human disease called IPEX, results in severe autoimmune pathology. Hence FoxP3+ Tregs are critical for the establishment and maintenance of immune homeostasis throughout the body downstream of gut exposure to beneficial and commensal microbes. This leads to a widely accepted model in which gut antigens directly induce FoxP3+ Tregs, and that these responding cells are necessary for immune suppression mediated by canonical inhibitory cytokines like IL-10 and TGFβ (Johnson et al, Glycobiology, Vol 28, Issue 1, Pages 50-58)

Not all therapies against psoriasis, ankylosing spondylitis, RA, too high levels of fungi in the intestine, and/or enhanced levels of innate immune responses in intestinal mucosa work as efficient for all patients. Accordingly there is a continuous need for new and additional compositions that can be used against such diseases. Hence there is a need for compositions that have an effect on the balance between regulatory cells and Th17 cells.

It is desired to identify compounds that may modulate immune response, in particular to identify compounds that dampen inflammatory responses and clear out inflammation after an immune-response to a pathogen.

It is also desired to have food compositions comprising one or more of such compounds that can help in the prevention of psoriasis, ankylosing spondylitis, RA, too high levels of fungi in the intestine, and/or enhanced levels of innate immune responses in intestinal mucosa.

It is a further desire that such compound(s) is/are considered safe, preferably have a GRAS status (Generally Recognized As Safe).

It is an objective of the present invention to provide a composition that addresses at least one of the aforementioned desires and or needs.

Peripheral blood mononuclear cells (PBMC) are widely used in immunogenicity predictions and toxicology applications. PBMCs give selective responses to the immune system and are the major cells in the human body immunity. The type of response being dependent on the type of stimulation. PBMCs are widely used as a model system to in vitro experiments to predict the effect of a composition in vivo (Wullner et al Clin Immunology 2010, vol 137 pp 5-14; Tapi-Calle et al Vaccines 2019 vol 7, 181).

WO 98/43494 concerns the analysis of a large number of human milk samples to determine appropriate average levels of nine important milk oligosaccharides and to arrive at the preparation of a synthetic infant formulation containing these oligosaccharides near the naturally occurring levels found in human breast milk.

Human Milk Oligosaccharides (HMOs) are ingredients of human milk that can be absorbed from the intestine and have an effect on the immune system in circulation. HMOs have been shown to prevent adhesion of several potential pathogens to epithelial surfaces in the intestine and other organs by acting as decoy receptors for bacterial pathogens like Campylobacter or E. coli. HMOs can also have effects on viral pathogens as rotavirus, norovirus, and HIV. In addition to effects on intestinal pathogens, HMOs have been suggested to also play a role in infections from respiratory viruses. Moreover, HMOs are known to dampen inflammatory responses and clear out inflammation after an immune-response to a pathogen. (Triantis V, Bode L, van Neerven R J J; Front Pediatr. 2018 Jul. 2; 6:190. doi: 10.3389/fped.2018.00190. eCollection 2018. Immunological Effects of Human Milk Oligosaccharides).

HMOs have antimicrobial and immunomodulatory actions (Comstock et al J Nutrition, 2017 147(6) Pages 1041-1047, https://doi.org/10.3945/jn.116.243774. Free human milk oligosaccharides (HMO) are among the most abundant components in human milk, after water and lactose. These are carbohydrates with a degree of polymerization from 3 to 32, composed of five monomers: D-glucose (Glc), D-galactose (Gal), N-acetylglucosamine (GlcNAc), L-fucose (Fuc) and N-acetylneuraminic acid (Neu5Ac, or sialic acid). The combinatory potential of structural isomers is high and HMO represent a large catalogue of complex carbohydrates. Human milk oligosaccharides carry lactose (Galβ1-4Glc) at the reducing end, which can be elongated by the addition of β1-3- or β1-6-linked lacto-N-biose (Galβ1-3GlcNAc-, type 1 chain) or N-acetyllactosamine (Galβ1-4GlcNAc-, type 2 chain). Lactose or the elongated oligosaccharide chain can be fucosylated in α1-2, α1-3, or α1-4 linkage and/or sialylated in α2-3 or α2-6 linkage. More than 150 different HMOs have been identified thus far. Most HMOs are found uniquely in human milk, from which they can be isolated. Alternatively, they can be synthesized using strategies to generate HMOs through chemoenzymatic synthesis, microbial metabolic engineering, and isolation from human donor milk or dairy streams (L. Bode et al Nutr Rev 2016 74(10) pp 635-644.).

Bovine milk is a potentially excellent source of commercially viable analogs of these unique molecules. However, bovine milk has a much lower concentration of these oligosaccharides than human milk, and the majority of the molecules are simpler in structure than those found in human milk. Consequently, individual HMO are isolated from human milk, with the disadvantage that it is available in limited amounts only. Alternatively, chemical and enzymatical synthesis of individual HMO is pursued. This resulted in few HMO being available in large quantities because of the difficulties that need to be overcome in the carbohydrate syntheses like obtaining the right chirality for each carbon atom and obtaining the right linkage between all monosaccharide units present in the oligosaccharide.

It is likely that not all individual HMO have the same biological effect. Consequently, it is of interest to know which HMO is/are responsible for certain immunological effect. In that way, not all HMOs need to be present in order to pursue an effect caused by the total pool of HMO.

### SUMMARY OF THE INVENTION

It was surprisingly found that LNFP-III and LSTa together had a similar gene regulating effect on peripheral blood mononuclear cells (PBMC) as a composition consisting of all HMOs present in human milk, as isolated from a collection of mother's milk. In addition it was found that that all the genes regulated by DSLNT were also regulated either by LNFP-III or by LSTa, so a combination of LSTa and LNFP-III together regulates the same genes as DSLNT.

LSTa, LNFP-III, and DSLNT caused and increase in the expression of FoxP3 just like the total pool of HMO (i.e. a mixture of all HMOs isolated from human milk). Fox P3 is a transcription factor that drives regulatory T cell (Treg) function and is a marker for regulatory T cells. FoxP3 causes production of IL10 which is involved in immune regulation.

Similarly, LSTa, LNFP-III and DSLNT caused an increase in the expression of IL10, just like the total pool of HMO. IL10 is a cytokine that is produced by regulatory T cell and is central in dampening inflammatory responses and clearing out inflammation after an immune response to a pathogen.

Surprisingly, 2′FL (2′-fucosyl lactose) the most abundant HMO present in the total pool of HMO had no effect on the expression of FoxP3 or IL10.

Because the increase in the expression of FoxP3 and IL10 was lower for DSLNT as compared to LSTa and LNFP-III, the invention thus relates to a synthetic composition, comprising LSTa and LNFP-III or a pharmaceutically acceptable salt, solvate or ester thereof, preferably a biologically effective amount of both LSTa and LNFP-III.

The composition of the invention may be used in therapeutic and non-therapeutic treatments. Non-therapeutic treatments are considered cosmetic treatments such as maintaining or keeping a healthy body weight.

The composition may be used in the modulation of one or more selected from the group consisting of
i. expression of FoxP3, preferably increased expression;
ii. expression of Th17, preferably increased expression;
iii. expression of IL10, preferably increased expression;
iv. modulate immune response; preferably to dampen inflammatory responses and clear out inflammation after an immune-response to a pathogen;
v. prevention of psoriasis or reduce the severity of psoriasis,
vi. prevention of ankylosing spondylitis or reduce the severity thereof;
vii. prevention of Rheumatoid Arthritis (RA) or reduce the severity thereof
viii. reduce the levels of fungi in the intestine, and
ix. enhance the innate immune responses in intestinal mucosa.

The invention also relates to a food product, in particular an infant formula comprising the synthetic composition of the invention. In another aspect the invention relates to the use of the synthetic composition of the invention for use in medicine.

In yet another aspect the invention relates to the synthetic composition of the invention for use in the non-therapeutic modulation of one or more selected from the group consisting of upregulating FoxP3, upregulating IL10, upregulation regulatory T cells (Tregs), and downregulation immune responses. In still another aspect the invention relates to the synthetic composition of the invention for use in the treatment of inflammatory diseases, such as ameliorating the effect of an inflammatory disease.

### DESCRIPTION OF FIGURES

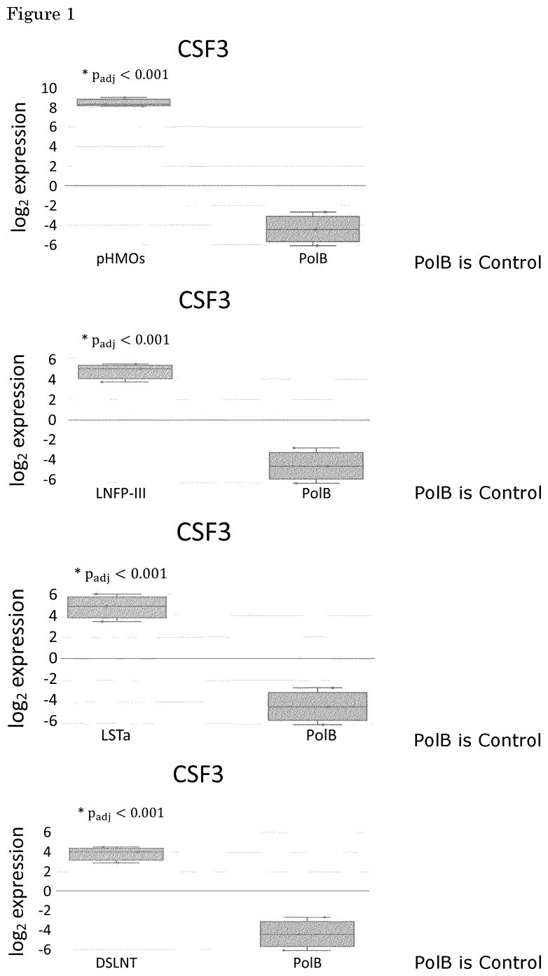

In FIGS. 1 to 4 the expression of CSF3, CD25 (IL2RA), IL12B and IL23A in PBMC is shown in FIGS. 1 to 4, respectively, as regulated by the pool of HMOs as isolated from human milk (pHMOs), LNFP-III, LSTa and DSLNT. All of these genes are involved in Th17 and Treg cells differentiation.

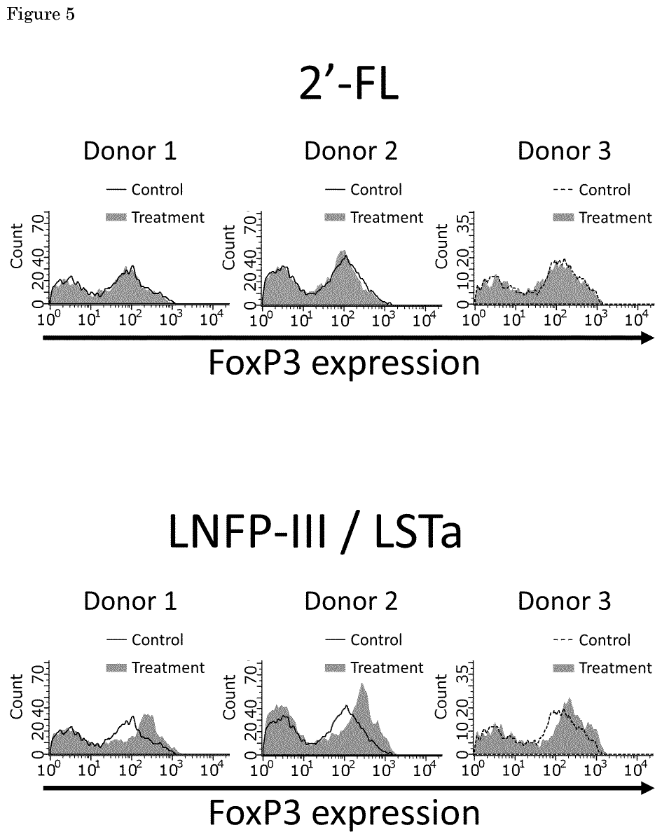

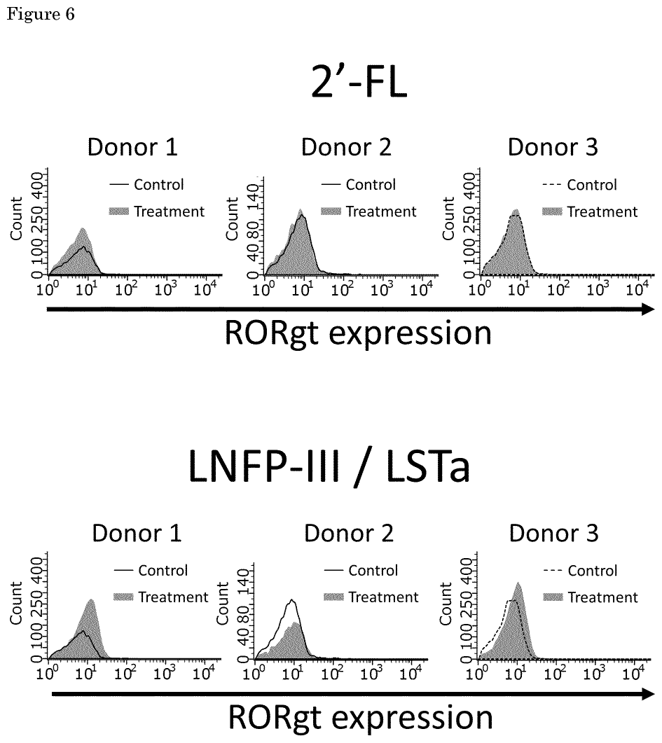

An increase in the expression of FoxP3 (FIG. 5) and RORγT (FIG. 6) is shown for cells treated with a mixture of LNFP III and LSTa, Cells treated with 2′FL showed a FoxP3 and RORγT expression similar to the control. In FIG. 6, RORgT stands of RORγT.

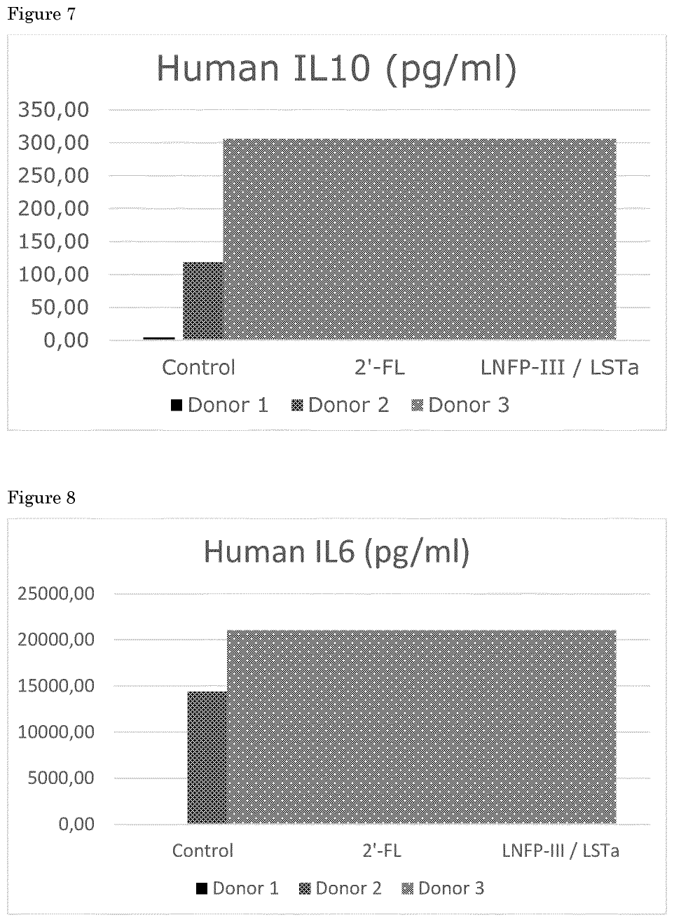

In FIG. 7 the expression of IL10 is shown for a mixture of LNFP III and LSTa as compared to control and to 2′FL.

In FIG. 8, the expression of IL6 is shown for a mixture of LNFP III and LSTa as compared to control and to 2′FL.

### DETAILED DESCRIPTION OF THE INVENTION

The term “treatment”, in relation a given disease or disorder, includes, but is not limited to, inhibiting the disease or disorder, for example, arresting the development of the disease or disorder; relieving the disease or disorder, for example, causing regression of the disease or disorder; or relieving a condition caused by or resulting from the disease or disorder, for example, relieving, preventing or treating symptoms of the disease or disorder.

The term “prevention” in relation to a given disease or disorder means preventing the onset of disease development if none had occurred, preventing the disease or disorder from occurring in a subject that may be predisposed to the disorder or disease but has not yet been diagnosed as having the disorder or disease, and/or preventing further disease/disorder development if already present.

It is also to be understood that this invention is not limited to the specific embodiments and methods described herein, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a”, “an,” and “the” comprise plural referents unless the context clearly indicates otherwise. For example, reference to a component in the singular is intended to comprise a plurality of components.

It will be understood that within this disclosure, any reference to a weight, weight ratio, and the like pertains to the dry matter, in particular the dry matter of the composition.

Unless defined otherwise, all technical and scientific terms used herein generally have the same meaning as commonly understood by one of ordinary skill in the art to which this invention belongs.

As used herein, the term “comprising”, which is synonymous with “including” or “containing”, is open-ended, and does not exclude additional, unrecited element(s), ingredient(s) or method step(s), whereas the term “consisting of” is a closed term, which excludes any additional element, step, or ingredient which is not explicitly recited.

Throughout this application, where publications are referenced, the disclosures of these publications in their entireties are hereby incorporated by reference into this application to more fully describe the state of the art to which this invention pertains

The term “subject” as used herein refers to a human, that is treatable by the method of the invention. The term “subject” refers to both the male and female sex unless one sex is specifically indicated. The human subject can be an infant, a juvenile, an adolescent, an adult or an elderly subject. The human subject can have an age of between 0-3 months, 0-6 months, 3-6 months, 0-12 months, 6-12 months, 12-24 months, 12-36 months, it can have an age of up to 5 years, up to 10, 12, 15, 20, or 30 years; or an age ≥30 years such as ≥40 year, ≥45 years, ≥50 years, ≥55 years, ≥60 years, ≥65 years, ≥70 years, ≥75 years, ≥80 years or even ≥85 years.

In embodiments of the invention the human subject is at least 18 years of age, e.g. at least 25 years, at least 30 years, at least 35 years, at least 40 years, at least 45 years, at least 50 years, at least 55 years, at least 60 years or at least 65 years of age. There is no particular upper limit although in practice, human subjects treated in accordance with the invention will typically be at most 100 years of age, e.g. at most 95 or at most 90 years of age.

As used herein, the term D-Gal refers to D-galactopyranose. The term D-GlcNAc refers to D-(Acetylamino)-2-deoxy-glucopyranose. L-Fuc refers to L-fucopyranose. D-Glc refers to D-glucopyranose. The “a” or “13” directly following the monosaccharide abbreviation (e.g. Glc, Gal, Fuc) indicate the chirality of the anomeric carbon. So, D-Gal-β1→4-D-Glc represents a lactose moiety i.e. a β-D-galactopyranose linked to the 4 position of D-glucose, with a β1→4 linkage. Likewise, Neu5Ac-α-2→3-D-Gal represents an N-acetyl-D-neuraminic acid (i.e. 5-acetamido-3,5-dideoxy-D-glycero-D-galacto-non-2-ulopyranosonic acid) residue linked with its anomeric carbon (i.e. carbon 2) in alpha configuration to the 3 position of a D-galactose residue (i.e. an α-2→3 linkage). If there is no number specified at the right-hand side of the linkage arrow, then the linkage may be to any free OH-group of the monosaccharide residue indicated, except for the anomeric OH.

“Effective amount” or “therapeutically effective amount” as used herein, refers to an amount of LNFP-III and LSTa, or a composition thereof further comprising DSLNT, that is effective in producing the desired therapeutic, ameliorative, inhibitory, non-therapeutic or preventative effect when administered to a patient suffering from a condition. An effective amount can refer to each individual agent alone or to the combination as a whole, wherein the amounts of all agents administered are together effective, but wherein the component agent of the combination may not be present individually in an effective amount. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. Preferably, the solvate is a hydrate. “Hydrate” is a solvate wherein the solvent molecule is H₂O.

The term “infant,” as used herein, unless otherwise specified, refers to a human 36 months of age or younger. The term “toddler,” as used herein, unless otherwise specified, refers to a subgroup of infant that is 12 months of age to 36 months of age. The term “child,” as used herein, unless otherwise specified, refers to a human 3 years of age to 18 years of age. The term “adult,” as used herein, unless otherwise specified, refers to a human 18 years of age or older. The term “synthetic composition” as used herein refers to a composition which is artificially prepared and preferably means a composition comprising at least one compound that is produced ex vivo chemically and/or biologically, e.g. by means of chemical reaction, enzymatic reaction or recombinantly, or purified by humans. The synthetic composition of the invention is not identical with a naturally occurring composition. The synthetic composition of the invention typically comprises one or more compounds, advantageously HMOs, but may further include other ingredients like protein, fat, minerals or vitamins. The synthetic composition of the invention is not milk from an animal, e.g. it is not mother's milk or cow milk.

So, in a first aspect the invention relates to a synthetic composition comprising a carbohydrate component, a fat component and a protein component, wherein the carbohydrate component comprises lacto-N-fucopentaose III (LNFP III) and sialyllacto-N-tetraose a (LSTa), preferably wherein the weight ratio between LNFP III and LSTa is between 1:100 and 100:1 This composition may optionally further comprise disialyllacto-N-tetraose (DSLNT).

As used herein, “Lacto-N-fucopentaose III” (LNFP III or LNPF-III) refers to β-D-Gal-(1→4)-[α-L-Fuc-(1→3)]-β-D-GlcNAc-(1→3)-β-D-Gal-(1→4)-D-Glc; CAS Number 25541-09-7); “sialyllacto-N-tetraose a” (LSTa) refers to α-Neu5Ac-(2→3)-β-D-Gal-(1→3)-β-D-GlcNAc-(1→3)-β-D-Gal-(1→4)-Glc; CAS Number 64003-58-5); and disialyllacto-N-tetraose (DSLNT) refers to α-Neu5Ac-(2→3)-β-D-Gal-(1→3)-[α-Neu5Ac-(2→6)]-β-D-GlcNAc-(1→3)-β-D-Gal-(1→4)-Glc; CAS Number 61278-38-4). LNFP III, LSTa and DSLNTare Human Milk Oligosaccharide (HMO) and may be obtained from commercial suppliers such as Dextra Laboratories (Reading, UK), Prozyme (Hayward, CA), Sigma Aldrich, Jennewein, or others. Alternatively, it may be synthesized using conventional organic chemistry methods, or it may be isolated from milk, e.g human milk using methods known in the art. Briefly, isolation from (human) milk may be done by obtaining (human) milk from volunteers of (preterm) infants. After centrifugation of the milk, the lipid layer is removed and proteins precipitated from the aqueous phase by addition of ice-cold ethanol and subsequent centrifugation. Ethanol can be removed from the HMO-containing supernatant by roto-evaporation.

Oligosaccharides may then be separated using anion exchange chromatography, particularly high-pH anion exchange chromatography with pulsed amperometric detection, Prior separation of the neutral and acidic oligosaccharides may be required. Alternatively, HMO may be separated using reverse phase (RP) HPLC (Ruhaak et g, Advances in Analysis of Human Milk Oligosaccharides Article in American Society for Nutrition, Advances in Nutrition 3, 4065-4145, May 2012; DOI: 10.3945/an.112.001883 Source: PubMed).

The amount of LSTa is less than 15 wt % (dry weight), preferably less than 10 wt % (dry weight).

In a first embodiment, the weight ratio between LNFP III and LSTa in the composition of the invention is between 1:100 and 100:1, preferably between 1:50 and 50:1, more preferably between 1:10 and 10:1, most preferably between 1:5 and 5:1. In another embodiment the weight ratio between LNFP III and DSLNT in the composition of the invention is between 1:100 and 100:1, preferably between 1:50 and 50:1, more preferably between 1:10 and 10:1, most preferably between 1:5 and 5:1. In still another embodiment, the weight ratio LNFP III:LSTa:DSLNT in the composition of the invention is between 1:(0.01-100):(0.01-100), preferably between 1:(0.02-50):(0.02-50), more preferably between 1:(0.1-10):(0.1-10), most preferably between 1:(0.2-5):(0.2-5). In one embodiment, the amount of LNFP III in the composition of the invention is between 0.0001 wt % and 15 wt % of the dry weight of the composition, preferably between 0.001 and 10 wt %, more preferably between 0.01 and 5 wt %, even more preferably between 0.01 and 15 wt %, particularly preferably between 0.01 and 1 wt % of the dry weight of the composition.

In another embodiment, the amount of LSTa in the synthetic composition of the invention is between 0.0001 wt % and 15 wt % of the dry weight of the composition, preferably between 0.001 and 10 wt %, more preferably between 0.01 and 5 wt %, even more preferably between 0.01 and 15 wt %, particularly preferably between 0.01 and 1 wt % of the dry weight of the composition.

In still another embodiment, the amount of LNFP III and LSTa in the synthetic composition of the invention are each between 0.0001 wt % and 15 wt % of the dry weight of the composition, preferably each between 0.001 and 10 wt %, more preferably each between 0.01 and 5 wt %, even more preferably between 0.01 and 15 wt %, particularly preferably each between 0.01 and 1 wt % of the dry weight of the composition. In one embodiment the wt % of LNFP III and LSTa is about the same in the composition of the invention.

In yet another embodiment, the amount of DSLNT in the synthetic composition of the invention is between 0.0001 wt % and 15 wt % of the dry weight of the composition, preferably between 0.001 and 10 wt %, more preferably between 0.01 and 5 wt %, particularly preferably between 0.01 and 1 wt % of the dry weight of the composition.

In a preferred embodiment, the amount of LNFP III, LSTa and DSLNT in the synthetic composition of the invention are each between 0.0001 wt % and 15 wt % of the dry weight of the composition, preferably each between 0.001 and 10 wt %, even more preferably between 0.01 and 15 wt %, more preferably each between 0.01 and 5 wt %, particularly preferably each between 0.01 and 1 wt % of the dry weight of the composition.

In one embodiment, the composition of the invention is a liquid composition and the amount of LNFP III, LSTa and optionally of DSLNT are each between 1 and 10,000 mg/L of the composition, preferably each between 10 and 5000 mg/L, more preferably each between 40 and 4000 mg/L of the composition, most preferably each between 100 and 2500 mg/L.

In another aspect the composition of the invention further comprises a fat component, preferably wherein the fat component is a mixture comprising vegetable fat and milk fat, more preferably wherein the milk fat is bovine milk fat. In yet another aspect the composition of the invention further comprises a protein component. Preferably the composition of the invention further comprises a fat component and a protein component, preferably wherein the fat component is a mixture comprising vegetable fat and milk fat, more preferably wherein the milk fat is bovine milk fat.

In one embodiment the carbohydrate component in the composition of the invention is between 0.001 wt % and 15 wt % of the dry weight of the composition, preferably between 0.001 and 10 wt %, more preferably between 0.01 and 5 wt %. Preferably, the total amount of oligosaccharides with a degree of polymerisation of from 3 to and including 10 in the carbohydrate component in the composition of the invention is between 0.001 wt % and 30 wt %, preferably below 25 wt % of the dry weight of the composition, more preferably between 0.01 and 20 wt %, even more preferably between 0.1 and 15 wt % of the dry weight of the composition.

In one embodiment, the amount of LNFP III, LSTa and DSLNT in the composition of the invention is between 0.001 gram and 15 gram per serving, preferably between 0.001 and 10 gram, more preferably between 0.01 and 5 gram. Particularly preferably, the carbohydrate component in the composition of the invention with a degree of polymerization of from 3 to and including 10 excluding any optional non-digestible oligosaccharides (such as GOS, or FOS), is between 0.001 gram and 25 gram per serving, preferably between 0.001 and 20 gram, more preferably between 0.005 and 20 gram even more preferably between 0.01 and 10 gram per serving.

Generally, any source of protein, or fat that is suitable for use in nutritional products is also suitable for use in the protein component or fat component of the invention, provided that such macronutrients are also compatible with the essential elements in the carbohydrate component of the nutritional composition as defined herein.

In one embodiment, the synthetic composition of the invention is an infant formula, preferably, wherein the composition is a formula for children having an age selected from the group consisting of 0-6 months, 0-12 months, 6-12 months 12-24 months, 12-36 months and 24-36 months. More preferably, wherein the composition is a formula for children having an age of 0-6 months, 0-12 months, or 12-36 months. In another embodiment the composition is an adult formula. The terms “infant formula” or “infant nutritional product” as used herein are used interchangeably to refer to nutritional compositions that have the proper balance of macronutrients, micro-nutrients, and calories to provide sole or supplemental nourishment for and generally maintain or improve the health of infants, toddlers, or both. Infant formulas preferably comprise nutrients in accordance with the relevant infant formula guidelines for the targeted consumer or user population, an example of which would be the Infant Formula Act, 21 U.S.C. Section 350(a). Another example with guidelines for nutrients of an infant formula, in particular for a person of 0-12 months of age and for children up to 36 months old, may be found in the CODEX Alimentarius (CODEX STAN 72-1981), further referred to as the CODEX). Nutritional compositions for infants are commonly referred to as infant formula. When used as infant formula, the composition as used in the various aspects of the invention should contain the ingredients in the amounts as prescribed by the CODEX and, if needed, as prescribed by additional regulations of individual countries. An example of an ingredient list of an infant formula meeting the requirements of the EU, China and Codex can for example be found on www.frieslandcampinaingredients.com/15 at app/uploads/2019/04/PDS ELN Essentiale-Start-IF-110.pdf.

In certain embodiments, when the nutritional powder is formulated as an infant formula, the protein component is typically present in an amount of from 5% to 35% by weight of the infant formula (i.e., the dry weight), including from 10% to 30%, from 10% to 25%, from 15% to 25%, from 20% to 30%, from 15% to 20%, and also including from 10% to 16% by weight of the infant formula (i.e., the dry weight). The carbohydrate component is typically present in an amount of from 40% to 75% by weight of the infant formula (i.e., the dry weight), including from 45% to 75%, from 45% to 70%, from 50% to 70%, from 50% to 65%, from 50% to 60%, from 60% to 75%, from 55% to 65%, and also including from 65% to 70% by weight of the infant formula (i.e., the dry weight). The fat component is typically present in an amount of from 10% to 40% by weight of the infant formula (i.e., the dry weight), including from 15% to 40%, from 20% to 35%, from 20% to 30%, from 25% to 35%, and also including from 25% to 30% by weight of the infant formula (i.e., the dry weight).

In certain embodiments, when the nutritional powder is formulated as a pediatric formula, the protein component is typically present in an amount of from 5% to 30% by weight of the pediatric formula (i.e., the dry weight). The carbohydrate component is typically present in an amount of from 40% to 75% by weight of the pediatric formula, (i.e., the dry weight). The fat component is typically present in an amount of from 10% to 25% by weight of the pediatric formula, (i.e., the dry weight).

In one embodiment the composition of the invention is a food supplement, which may also be referred to as “dietary supplement” i.e. a manufactured product intended to supplement one's diet by taking a pill, capsule, tablet, powder or liquid. A supplement can provide nutrients in order to increase the quantity of their consumption. The class of nutrient compounds includes specific carbohydrates, vitamins, minerals, fiber, fatty acids, amino acids or combinations thereof.

Alternatively, the composition of the invention is a product aimed at adults, i.e. an adult formula. As used herein, the terms “adult formula” and “adult nutritional product” as used herein are used interchangeably to refer to nutritional compositions suitable for generally maintaining or improving the health of an adult. When the nutritional powder is formulated as an adult nutritional product, the protein component is typically present in an amount of from 5% to 35% by weight of the adult nutritional product, including from 10% to 25%, and including from 20% to 30% by weight of the adult nutritional product (dry weight). The carbohydrate component is typically present in an amount of from 40% to 80% by weight of the adult nutritional product, including from 50% to 75%, and also including from 60% to 75% by weight of the adult nutritional product. The fat component is typically present in an amount of from 0.5% to 20%, including from 1% to 15%, and also including from 15% to 20% by weight of the adult nutritional product.

Generally, any source of protein may be used so long as it is suitable for oral nutritional compositions and is otherwise compatible with any other selected ingredients or features in the nutritional composition. Non-limiting examples of suitable proteins (and sources thereof) suitable for use in the nutritional powders described herein include, but are not limited to, intact, hydrolyzed, or partially hydrolyzed protein, which may be derived from any known or otherwise suitable source such as milk (e.g., casein, whey), animal (e.g., meat, fish), cereal (e.g., rice, corn, wheat), vegetable (e.g., soy, pea, potato, bean), and combinations thereof. The protein may also include a mixture of amino acids (often described as free amino acids) known for use in nutritional products or a combination of such amino acids with the intact, hydrolyzed, or partially hydrolyzed proteins described herein. The amino acids may be naturally occurring or synthetic amino acids.

More particular examples of suitable protein (or sources thereof) used in the nutritional powders disclosed herein include, but are not limited to, whole cow's milk, partially or completely defatted milk, milk protein concentrates, milk protein isolates, nonfat dry milk, condensed skim milk, whey protein concentrates, whey protein isolates, acid caseins, sodium caseinates, calcium caseinates, potassium caseinates, legume protein, soy protein concentrates, soy protein isolates, pea protein concentrates, pea protein isolates, collagen proteins, potato proteins, rice proteins, wheat proteins, canola proteins, quinoa, insect proteins, earthworm proteins, fungal (e.g., mushroom) proteins, hydrolyzed yeast, gelatin, bovine colostrum, human colostrum, glycol macropeptides, mycoproteins, proteins expressed by microorganisms (e.g., bacteria and algae), and combinations thereof. The nutritional powders described herein may include any individual source of protein or combination of the various sources of protein listed above. In addition, the proteins for use herein can also include, or be entirely or partially replaced by, free amino acids known for use in nutritional products, non-limiting examples of which include L-tryptophan, L-glutamine, L-tyrosine, L-methionine, L-cysteine, taurine, L-arginine, L-carnitine, and combinations thereof.

The carbohydrate or source of carbohydrate suitable for use in the composition disclosed herein may be simple, complex, or variations or combinations thereof. Generally, the carbohydrate may include any carbohydrate or carbohydrate source that is suitable for use in oral nutritional compositions and is otherwise compatible with any other selected ingredients or features in the nutritional powder. Non-limiting examples of carbohydrates suitable for use in the nutritional powders described herein include, but are not limited to, polydextrose, maltodextrin; hydrolyzed or modified starch or cornstarch; glucose polymers; corn syrup; corn syrup solids; sucrose; glucose; fructose; lactose; high fructose corn syrup; honey; sugar alcohols (e.g., maltitol, erythritol, sorbitol); isomaltulose; sucromalt; pullulan; potato starch; and other slowly-digested carbohydrates; dietary fibers including, but not limited to, fructooligosaccharides (FOS), galactooligosaccharides (GOS), oat fiber, soy fiber, gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinogalactans, glucomannan, xanthan gum, alginate, pectin, low methoxy pectin, high methoxy pectin, cereal beta-glucans (e.g., oat beta-glucan, barley beta-glucan), carrageenan and psyllium, soluble and insoluble fibers derived from fruits or vegetables; other resistant starches; and combinations thereof. The nutritional powders described herein may include any individual source of carbohydrate or combination of the various sources of carbohydrate listed above.

The fat or source of fat suitable for use in the nutritional powders described herein may be derived from various sources including, but not limited to, plants, animals, and combinations thereof. Generally, the fat may include any fat or fat source that is suitable for use in oral nutritional compositions and is otherwise compatible with any other selected ingredients or features in the nutritional powder. Non-limiting examples of suitable fat (or sources thereof) for use in the nutritional powders disclosed herein include coconut oil, fractionated coconut oil, soy oil, high oleic soy oil, corn oil, olive oil, safflower oil, high oleic safflower oil, medium chain triglyceride oil (MCT oil), high gamma linolenic (GLA) safflower oil, sunflower oil, high oleic sunflower oil, palm oil, palm kernel oil, palm olein, canola oil, high oleic canola oil, marine oils, fish oils, algal oils, borage oil, cottonseed oil, fungal oils, eicosapentaenoic acid (EPA), docosahexaenoic acid (DHA), arachidonic acid (ARA), conjugated linoleic acid (CLA), alpha-linolenic acid, rice bran oil, wheat bran oil, interesterified oils, transesterified oils, structured lipids, and combinations thereof. Generally, the fats used in nutritional powders for formulating infant formulas and pediatric formulas provide fatty acids needed both as an energy source and for the healthy development of the infant, toddler, or child. These fats typically comprise triglycerides, although the fats may also comprise diglycerides, monoglycerides, and free fatty acids. Fatty acids provided by the fats in the nutritional powder include, but are not limited to, capric acid, lauric acid, myristic acid, palmitic acid, palmitoleic acid, stearic acid, oleic acid, linoleic acid, alpha-linolenic acid, ARA, EPA, and DHA. The nutritional powders can include any individual source of fat or combination of the various sources of fat listed above. Preferably, the fat is a mixture of vegetable fat and milk fat such as obtained from milk from a mammal like cow, sheep, goat, mare, or camel. More preferably, wherein the milk fat is bovine milk fat. Mixtures of different types of fat are preferred because they help to provide different fatty acids and better resemble the type of linkage between the glycerol moiety and the fatty acid moiety in the fat, when compared to human mother's milk.

In certain embodiments, the nutritional powders described herein may further comprise other optional ingredients that may modify the physical, chemical, hedonic, or processing characteristics of the products or serve as additional nutritional components when used for a targeted population. Many such optional ingredients are known or otherwise suitable for use in other nutritional products and may also be used in the nutritional powders described herein, provided that such optional ingredients are safe and effective for oral administration and are compatible with the essential and other ingredients in the selected product form. Non-limiting examples of such optional ingredients include preservatives, antioxidants, emulsifying agents, buffers, additional nutrients as described herein, colorants, flavors (natural, artificial, or both), thickening agents, flow agents, anti-caking agents, and stabilizers.

In certain embodiments, the nutritional powders further comprise minerals, non-limiting examples of which include calcium, phosphorus, magnesium, iron, zinc, manganese, copper, sodium, potassium, molybdenum, chromium, selenium, chloride, and combinations thereof.

In certain embodiments, the nutritional powders further comprise vitamins or related nutrients, non-limiting examples of which include vitamin A, vitamin D, vitamin E, vitamin K, thiamine, riboflavin, pyridoxine, vitamin B12, niacin, folic acid, pantothenic acid, biotin, vitamin C, choline, inositol, salts and derivatives thereof, and combinations thereof.

The composition of the invention may be powder (or powder product), a pill, a capsule, a pod or a liquid (or liquid product). The term “pod” as used herein, unless otherwise specified, refers to a sealable, re-sealable or sealed container having an internal volume capable of containing a solid, powder, or liquid formulation that, when mixed with liquid, yields a liquid product suitable for human consumption.

The term “liquid product” as used herein, unless otherwise specified, refers to the reconstituted nutritional powder.

The term “nutritional powder” as used herein, unless otherwise specified, refers to nutritional products that are solids or semisolids in the form of particles that are generally flowable or scoopable. A nutritional powder is usually reconstituted by addition of water or another liquid to form a liquid nutritional composition prior to administration to (e.g., providing to or consumption by) an individual. As discussed below, in certain embodiments disclosed herein, the nutritional powders comprise at least one of a source of protein, a source of carbohydrate, and a source of fat.

The terms “reconstitute,” “reconstituted,” and “reconstitution” as used herein, unless otherwise specified, are used to refer to a process by which the nutritional powder is mixed with a liquid, such as water, to form an essentially homogeneous liquid product. Once reconstituted in the liquid, the ingredients of the nutritional powder may be any combination of dissolved, dispersed, suspended, colloidally suspended, emulsified, or otherwise blended within the liquid matrix of the liquid product. Therefore, the resulting reconstituted liquid product may be characterized as any combination of a solution, a dispersion, a suspension, a colloidal suspension, an emulsion, or a homogeneous blend.

The term “serving” as used herein, unless otherwise specified, is any amount of a composition that is intended to be ingested by a subject in one sitting or within less than about one hour. The size of a serving (i.e., “serving size”) may be different for diverse individuals, depending on one or more factors including, but not limited to, age, body mass, gender, species, or health. For a typical human child or adult, a serving size of the compositions disclosed herein is from about 25 mL to 1,000 mL, or when taken as a solid product from 30 g to 250 g, such as from 100 to 15 g. For a typical human infant or toddler, a serving size of the compositions disclosed herein is from about 5 mL to about 250 mL.

In one embodiment the synthetic composition is a food product selected from the group consisting of confectionary products; nutritionally complete food products; desserts, in particular a pudding, yoghurt, custard, vla, ice-cream, or milk-shake; beverages, in particular a fruit juice or milk; breakfasts, such as porridge, cereals; soups; and sauces. A preferred food product is a confectionary product selected from the group of food-bar (such as granola bars, candy bars), sweeties and cookies.

The composition of the invention may further comprise human milk oligosaccharides (HMO, or HMOs) other than LNFP-III, LSTa and DSLNT. HMOs are oligosaccharides that occur in human milk. Human milk oligosaccharides (HMOs) are a key constituent of human milk. They are a structurally and biologically diverse group of complex indigestible carbohydrates. To date, more than 150 different oligosaccharides have been identified, varying in size from 3 to 22 monosaccharide units. The most common HMOs are the neutral fucosylated and non-fucosylated oligosaccharides. The quantity and structure of these HMOs differ significantly among women and is dependent upon Secretor and Lewis blood group status (L. Bode, J. Nutr. 136: 2127-2130, 2006.). In one embodiment, the composition as used in the aspects of the invention comprises one or more HMOs.

The HMOs of human milk are composed of various monosaccharides, namely glucose, galactose, fucose, N-acetylglucosamine and sialic acids (N-acetylneuraminic acid). The sugar fucose is an unusual molecule in that it has the L-configuration, whereas the other sugar molecules in the body have the D-configuration. The structure of HMOs is a lactose unit which may be elongated with one or more galactose and/or N-acetylglucosamine residues (core structure). The HMO core structure may be decorated with one or more fucose residues (i.e. fucosylated HMO) and with one or more sialic acid units (i.e. sialylated HMO). A HMO may also be fucosylated and sialylated. In one embodiment, the HMO in the composition of the invention is selected from one or more the group consisting of core HMO, sialylated HMO, and fucosylated HMO. Nearly 200 HMOs have been identified from human milk. Fucosylated HMOs were found to be the most prominent component (˜77%), while sialylated HMOs accounted for about 16% of the total abundance of HMOs. The fucosylated HMOs are neutral molecules, while the sialylated HMOs are acidic. In human milk, the most abundant HMO is 2′-fucosyllactose (a neutral trisaccharide composed of L-fucose, D-galactose, and D-glucose units, linked Fuc(α1-2)Gal(β1-4)Glc; CAS Nr 41263-94-9), with a concentration of about 2 g/l (Adams et al; 2018, Nutrafoods pp 169-173). Preferred HMOs are 3′-Sialyllactose (3′SL); 6′-Sialyllactose (6′SL); 2′-Fucosyllactose (2′FL); 3-Fucosyllactose (3-FL); lacto-N-tetraose (LNT), lacto-N-neotetraose (LNnT), LNFP II, LNFP III, and disialyllacto-N-tetraose (DSLNT). Particularly preferred nutritional compositions include at least 2′FL. HMOs can be obtained using methods known to those of skill in the art. For example, HMOs can be purified from human milk. Individual HMOs can be further separated using methods known in the art such as capillary electrophoresis, HPLC (e.g., high-performance anion-exchange chromatography with pulsed amperometric detection; HPAEC-PAD), and thin layer chromatography. See, e.g., U.S. Patent Application No. 2009/0098240. Alternately, enzymatic methods can be used to synthesize HMOs. Another method to manufacture HMO's is via biosynthesis in engineered bacteria. For example, a method of preparing 2′-FL is disclosed in WO 2012/112777. Alternatively, 2′-FL is commercially available e.g. from FrieslandCampina, or others.

The composition of the invention may further comprise one or more probiotics and/or one or more prebiotics. Probiotics and prebiotics are known in the art and are claimed to have beneficial effect on the subject's gut microbiome and to have a positive effect on the subject's health and/or wellbeing.

Non-digestible oligosaccharides are a class of prebiotics. In another embodiment of the invention, the composition comprises 0.25 to 20 wt. % non-digestible oligosaccharides based on dry weight of the composition, preferably wherein the non-digestible oligosaccharides are selected from one or more of galacto-oligosaccharides (GOS), and fructo-oligosaccharides (FOS), more preferably, wherein the non-digestible oligosaccharides are galacto-oligosaccharides. In other embodiments the minimum amount of non-digestible oligosaccharides is at least 1 wt % based on dry weight of the composition, such as at least 5 wt %. In yet another embodiment, the maximum amount of non-digestible oligosaccharide is 25 wt % based on dry weight of the composition, preferably less than 20 wt %, more preferably less than 15 wt %. Preferably, the composition comprises between 0.25 and 20 wt % GOS, more preferably between 1 and 10 wt % GOS, based on dry weight of the composition. GOS and FOS are commercially available and FOS may include inulin.

Probiotics are live microorganisms promoted with claims that they provide health benefits when consumed, generally by improving or restoring the gut flora. Probiotics are well known in the art and examples include Saccharomyces boulardii (a yeast) and bacteria in the Lactobacillus and Bifobacterium families of microorganisms. Lactobacillus acidophilus is the probiotic that is found in some yogurts.

The synthetic composition comprising the compound(s) of formula 1 may also be used for use in medicine, as a medicament. Preferably for the indications cited herein. Preferably for use in a human subject.

The composition of the invention may be used as an anti-inflammatory agent and/or for use in one or more of: upregulating FoxP3, upregulating IL10, upregulation regulatory T cells (Tregs), and downregulation immune responses. In one embodiment the composition of the invention may be used in a method of treating inflammatory disease, for example in one or more selected from the group consisting of protection of mucosal surfaces, to upregulate Th17, to activate immune response, to upregulating ROR γt, and, to upregulating IL6. IL6 is a driving force for producing Th17, ROR γt is a transcription factor for Th17. Th17 gives better protections against pathogens (viruses and bacteria) in mucosal surfaces/intestinal airway pathogens/infections.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: percent, “parts of,” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies, mutatis mutandis, to normal grammatical variations of the initially defined abbreviation; and, unless expressly stated to the contrary, measurement of a property is determined by the same technique as previously or later referenced for the same property.

In one embodiment, the composition of the invention comprises less than 0.01 wt % (as determined on dry matter) of one or more of TFLNH, DF-para-LNH, DF-para-LNnH, FS-LNH I, FS-LNH II, FS-LNnH1 or FDS-LNH II. Preferably, less than 0.001 wt, more preferably less than 0.0001 wt %, as determined on dry matter.

TFLNH is herein defined as trifucosyllacto-N-hexaose, β-D-Gal-(1→4)-[α-L-Fuc-(1→3)]-β-D-GlcNAc-(1→6)-[α-L-Fuc-(1→2)-β-D-Gal-(1→3)-[α-L-Fuc-(1→4)]-β-D-GlcNAc-(1→3)]-β-D-Gal-(1→4)-D-Glc; DF-para-LNH is defined as Difucosyl-para-lactohexaose, β-D-Gal-(1→3)-[α-L-Fuc-(1→4)]-β-D-GlcNAc-(1→3)-β-D-Gal-(1→4)-[α-L-Fuc-(1→3)]-D-GlcNAc-(1→3)]-β-D-Gal-(1→4)-D-Glc; DF-para-LNnH is defined as Difucosyl-para-lacto-N-neohexaose, β-D-Gal-(1→4)-[α-L-Fuc-(1→3)]-β-D-GlcNAc-(1→3)-β-D-Gal-(1→4)-[α-L-Fuc-(1→3)]-D-GlcNAc-(1→3)]-β-D-Gal-(1→4)-D-Glc; FS-LNH I is defined as Fucosyl-sialyl-lacto-N-hexaose I, β-D-Gal-(1→4)-[α-L-Fuc-(1→3)]-β-D-GlcNAc-(1→6)-[β-D-Gal-(1→3)-[α-D-Neu5Ac-(2→6)]-β-D-GlcNAc-(1→3)]-β-D-Gg-(1→4)-D-Glc, FS-LNH II is defined as Fucosyl-sialyl-lacto-N-hexaose II, β-D-Gal-(1→4)-[α-L-Fuc-(1→3)]-β-D-GlcNAc-(1→6)-N-D-Neu5Ac-(2→6)-β-D-Gal-(1→3)-β-D-GlcNAc-(1→3)]-β-D-Gal-(1-4)-D-Glc; FS-LNnH1 is defined as Fucosylsialyllacto-N-neohexaose I, β-D-Gal-(1→4)-[α-L-Fuc-(1→3)]-β-D-GlcNAc-(1→6)-[α-D-Neu5Ac-(2→3)-β-D-Gal-(1→3)-β-D-GlcNAc-(1→3)]-β-D-Gal-(1→4)-D-Glc; and FDSLNH II is defined as Fucosyl-disialyllacto-N-hexaose II, β-D-Gal-(1→4)-[α-L-Fuc-(1→3)]-β-D-GlcNAc-(1→6)-N-D-Neu5Ac-(2→3)-β-D-Gal-(1→3)-[α-D-Neu5Ac-(2→6)]-β-D-GlcNAc-(1→3)]-β-D-Gal-(1→4)-D-Glc.

The invention is hereinafter illustrated with reference to the following, non-limiting, examples.

### EXAMPLES

PBMC Cell Isolation and HMO Incubations

Peripheral blood mononuclear cells (PBMCs) were isolated from a buffy coat using Sepmate tubes. The cells were diluted to a concentration of 1*10⁷ cells per ml. The PBMC's were seeded in 6 wells plates and treated with 0.5 g/l of each HMO in PBMC culturing medium for 6 h prior to RNA isolation. Each human milk oligosaccharide (HMO) was mixed with 100 ug/ml (microgram/milliliter) final concentration of Polymyxin B to ensure neutralization of possible endotoxin contamination of HMOs, while equal amounts of Polymyxin B were used as a control. As a positive control HMOs isolated from pooled human milk sample were used. Single HMOs were purchased from BOC Sciences.

RNA Sequencing

2500 ng RNA, isolated from PBMCs of 3 donors, was translated into cDNA, using the quantitect reverse transcription kit (Qiagen). RNA sequencing and analysis was performed by use of the NOVOGENE platform according to standard procedures. Gene expression is shown for CSF3, CD25 (IL2RA), IL12B and IL23A as regulated by the pool of HMOs as isolated from human milk (pHMOs), LNFP-III, LSTa and DSLNT.

Intracellular Staining Transcription Factors

For intracellular staining PBMCs from 3 donors were incubated with the mentioned HMOs (0.5 g/1) for 24 h prior to analysis. In a 96-wells plate 5*10⁵ PBMCs were pelleted at 300 g for 4 minutes. Subsequently, the cells were blocked using 25 μL of blocking buffer (Human BD Fc Block™,BD) and incubated for 10 minutes at room temperature. Then, 25 μL of anti-CD4 FITC (555346, BD) and anti-CD3 APC (555335, BD) were added to each test. The cells were incubated 30 minutes on ice. After the cells were washed twice with Stain Buffer (FBS) (554657, BD) an intracellular staining for the transcription factors was performed by the Transcription Factor Buffer Set (562574, BD). FoxP3 PE (560852, BD) and RORγT PE (563081, BD) were used. Cells were analyzed with FACS (Easycyte 8HT). Expression of FoxP3 and RORγT was analyzed in the CD4-positive cells.

Cytokine Analysis

BD™ Cytometric Bead Array (CBA) Human Th1/Th2/Th17 Cytokine Kit (560484, BD) was used to measure Interleukin-6 (IL-6) and Interleukin-10 (IL-10) protein levels in supernatant following the manufacturer's instructions. After acquiring samples on the FACS (Easycyte 8HT), the FCAP Array™ software was used to determine cytokine concentrations.

### Example 1: RNA Sequencing in PBMCs and Expression of Important Genes in the Differentiation of Th17 and Treg Cells (FIGS. 1-4)

PBMC's from 3 donors were isolated from a buffy coat using Sepmate tubes. The cells were diluted to a concentration of 1*10⁷ cells per ml. The PBMC's were seeded in 6 wells plates and treated with 0.5 g/l of each HMO in PBMC culturing medium for 6 h prior to RNA isolation. Each HMO was mixed with 100 ug/ml final concentration of Polymyxin B to ensure neutralization of possible endotoxin contamination of HMOs, while equal amounts of Polymyxin B were used as a control. As a positive control HMOs isolated from pooled human milk sample were used. Single HMOs were purchased from BOC Sciences.

2500 ng RNA, isolated from PBMCs, was translated into cDNA, using the quantitect reverse transcription kit (Qiagen). RNA sequencing and analysis was performed by use of the NOVOGENE platform according to standard procedures. Gene expression was shown for CSF3, CD25 (IL2RA), IL12B and IL23A as regulated by the pool of HMOs as isolated from human milk (pHMOs), LNFP-III, LSTa and DSLNT.

The expression of CSF3, CD25 (IL2RA), IL12B and IL23A in PBMC is shown in FIGS. 1 to 4, respectively, when these PBMCs were treated with the total pool of HMOs (pHMOs), LNFP-III, LSTa and DSLNT. In each figure Polymyxin B was used as control. The relative expression of CSF3 was about 8.5 for the pHMOs, between 4 and 6 for LNFP III and LSTa, while DSLNT had a slightly lower expression (around 4), as shown in FIG. 1.

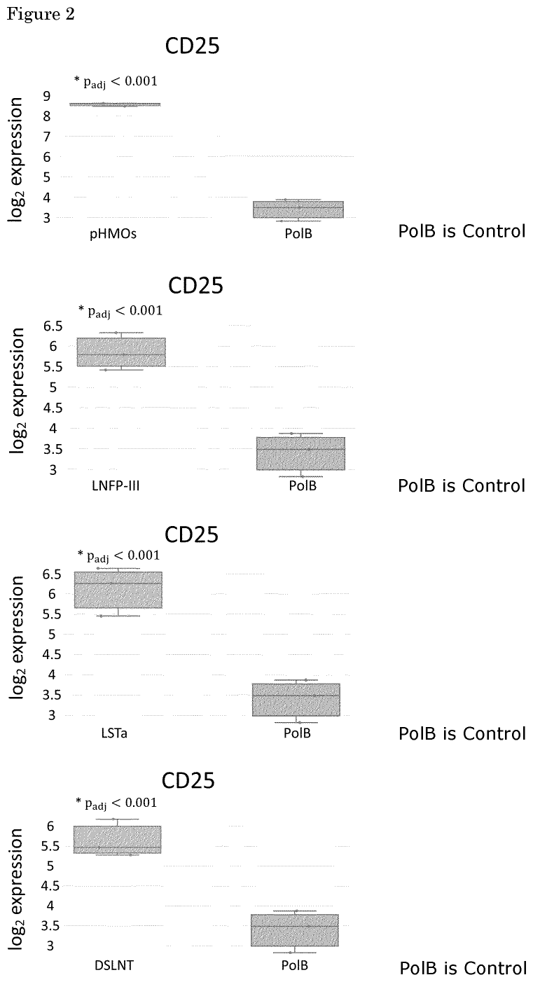

A similar trend was observed in the expression of CD25: pHMO around 8.5; LNFP III around 5.8; LSTa around 6.3; and DSLNT around 5.5 (FIG. 2).

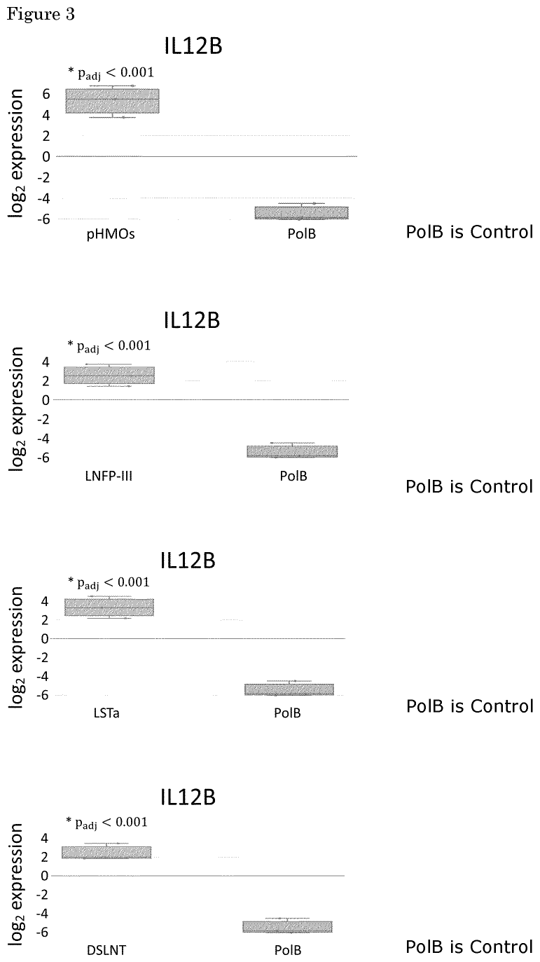

The expression of IL12B: pHMOs about 5.5; LNFP III about 2.5; LSTa about 3; and DSLNT around 2.5; as shown in FIG. 3.

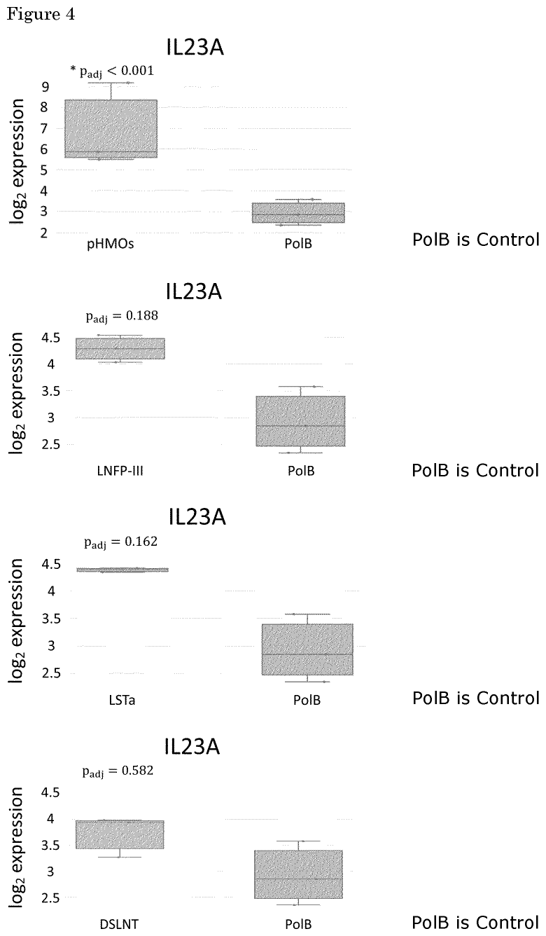

The expression of IL23A; pHMOs about 6; LNFP III about 4.3; LSTa about 4.4; and DSLNT between about 3.5 and 4.0 (FIG. 4).

These experiments show that LNFP III and LSTa show a similar expression of these genes as compared to the total pHMO. A similar—but slightly lower effect was shown for DSLNT. LNFP III, LSTa and DSLNT hence are shown to be effective in modifying gene expression of genes involved in the TH17 and Treg cell differentiation.

### Example 2: Expression of FoxP3 and RORγT in PBMCs (FIGS. 5, 6)

PBMC's from 3 donors were isolated from a buffy coat using Sepmate tubes. The cells were diluted to a concentration of 1*10⁷ cells per ml. The PBMC's were seeded in 6 wells plates and treated with 0.5 g/l of 2′-FL or a mix of LNFP-III with LSTa 1:1 w/v in PBMC culturing medium for 24 h. Cells incubated with no HMOs were used as control. For Fluorescence Activated Cell Sorting (FACS), 5*10⁵ PBMCs were pelleted at 300 g for 4 minutes in a 96-well plate.

Subsequently, the cells were blocked using 25 μL of blocking buffer (Human BD Fc Block™,BD) and incubated for 10 minutes at room temperature. Then, 25 μL of anti-CD4 FITC (555346, BD) and anti-CD3 APC (555335, BD) were added to each test. The cells were incubated 30 minutes on ice. After the cells were washed twice with Stain Buffer (FBS) (554657, BD) an intracellular staining for the transcription factors was performed by the Transcription Factor Buffer Set (562574, BD). FoxP3 PE (560852, BD) and RORγT PE (563081, BD) were used. Cells were analyzed with FACS (Easycyte 8HT). Expression of FoxP3 and RORγT was analyzed in the CD4-positive cells and is shown in FIGS. 5 and 6, respectively. Cells treated with 2′FL showed a FoxP3 and RORγT expression similar to the control, while the mixture of LNFP III and LSTa showed an increase in the expression of these two genes as reflected by a shift to the right of the peak of the curve as compared to the control. This effect was observed for all donors.

### Example 3: Expression of IL10 and IL6 in PBMCs (FIGS. 7, 8)

PBMC's from 3 donors were isolated from a buffy coat using Sepmate tubes. The cells were diluted to a concentration of 1*10⁷ cells per ml. The PBMC's were seeded in 6 wells plates and treated with 0.5 g/l of 2′-FL or a mix of LNFP-III with LSTa 1:1 w/v in PBMC culturing medium for 24 h. Cells incubated with no HMOs were used as control. The supernatants from these cultures were collected and BD™ Cytometric Bead Array (CBA) Human Th1/Th2/Th17 Cytokine Kit (560484, BD) was used to measure Interleukin-6 (IL-6) and Interleukin-10 (IL-10) protein levels in supernatant following the manufacturer's instructions. After acquiring samples on the FACS (Easycyte 8HT), the FCAP Array™ software was used to determine cytokine concentrations. As illustrated in FIG. 7, the expression of IL10 for cells treated with 2′FL was less than 3 pg/ml and similar to the control, while cells treated with the mixture of LNFP III and LSTa all showed a positive expression of IL10 with an average expression of IL10 of around 167 pg/ml.

The expression of IL6 is shown in FIG. 8. Also in this experiment, there was no expression of IL6 by cells treated with 2′FL, just like in the control. Cells treated with LNFP III and LSTa showed a positive expression of IL6—for all donors—with an average expression of 12500 pg/ml.

In a similar experiment it was shown that the mixture of LNFP III and LSTa upregulate IL12B, IL23A, CSF3 and CD25 (IL2RA).

So in still another embodiment, the composition of the invention may be used to upregulated the expression of one or more selected from the group consisting of IL12B, IL23A, CSF3 and CD25 (IL2RA). In yet another embodiment the invention relates to the use of the composition of the invention for the invention in the treatment or to ameliorate the effects of a low expression of any one or more of the genes which have an increased expression as exemplified in any of the examples (such as one or more of CSF3, CD25 (IL2RA), IL12B, IL23A, FoxP3, RORγT, IL6 and IL10).

1. A synthetic composition comprising a carbohydrate component, a fat component and a protein component, wherein the carbohydrate component comprises lacto-N-fucopentaose III (LNFP III) and sialyllacto-N-tetraose a (LSTa), and the amount of LSTa is less than 15 wt % of the dry weight of the composition.

2. The synthetic composition of claim 1, wherein the amount of LNFP III is between 0.0001 wt % and 15 wt % of the dry weight of the composition.

3. The synthetic composition of claim 1, wherein the amount of LSTa is between 0.0001 wt % and 15 wt % of the dry weight of the composition.

4. The synthetic composition of claim 1, wherein the amount of LNFP III and LSTa are each between 0.0001 wt % and 15 wt % of the dry weight of the composition.

5. The synthetic composition of claim 1, further comprising disialyllacto-N-tetraose (DSLNT).

6. The synthetic composition of claim 5, wherein the amount of DSLNT is between 0.0001 wt % and 15 wt % of the dry weight of the composition.

7. The synthetic composition of claim 5, wherein the amount of LNFP III, LSTa and DSLNT are each between 0.0001 wt % and 15 wt % of the dry weight of the composition.

8. The synthetic composition of claim 1, wherein the synthetic composition is a liquid composition and the amount of LNFP III, LSTa and optionally of DSLNT are each between 1 and 10,000 mg/L of the composition.

9. The synthetic composition of claim 1, wherein the fat component is a mixture comprising vegetable fat and milk fat.

10. The synthetic composition of claim 1, wherein the composition is an infant formula.

11. The composition of claim 1, wherein the composition is an adult formula or wherein the composition is a food supplement.

12. The synthetic composition of claim 1, further comprising one or more probiotics and/or one or more prebiotics.

13. A medicament comprising the synthetic composition of claim 1.

14. The synthetic composition of claim 1, wherein the synthetic composition is effective as an anti-inflammatory agent and/or has an effect of one or more of:
upregulating FoxP3,
upregulating IL10,
upregulation regulatory T cells (Tregs), and
downregulation immune responses.

15. A method of treating inflammatory disease in a subject, the method comprising administering to the subject an effective amount of the synthetic composition of claim 1.

16. The synthetic composition of claim 1, wherein the weight ratio between LNFP III and LSTa is between 1:100 and 100:1.

17. The synthetic composition of claim 5, wherein the weight ratio LNFP III:LSTa:DSLNT is between 1:(0.01-100):(0.01-100).

18. The synthetic composition of claim 6, wherein the amount of LNFP III, LSTa and DSLNT are each between 0.0001 wt % and 15 wt % of the dry weight of the composition.

19. The synthetic composition of claim 9, wherein the milk fat is bovine milk fat.

20. The synthetic composition of claim 10, wherein the composition is a formula for children having an age selected from the group consisting of 0-2 months, 0-4 months, 0-6 months, 0-12 months, 2-6 months, 4-6 months, 6-12 months, 12-24 months, 12-36 months and 24-36 months.
